(19) United States
(12) Patent Application Publication
(10) Pub. No.: US 2022/0370698 A1
(43) Pub. Date: Nov. 24, 2022

(54) AUTONOMOUS DIALYSIS CONTROL

(71) Applicant: National University of Singapore, Singapore (SG)

(72) Inventors: Yi-Chih CHENG, Kaohsiung (TW); Tzu-Yang LIN, Taoyuan (TW)

(21) Appl. No.: 17/774,539

(22) PCT Filed: Dec. 4, 2020
(86) PCT No.: PCT/SG2020/050720

§ 371 Date: May 5, 2022

(30) Foreign Application Priority Data
Dec. 4, 2019 (SG) 10201911641T

(51) Int. Cl. A61M 1/28

(52) U.S. Cl. CPC A61M 1/28; A61M 2205/3334; A61M 2205/3344; A61M 2205/331; A61M 2205/3313

(57) ABSTRACT

The invention relates to a system for dialysis, comprising a housing comprising a flow path along which dialysate passes between a subject and a dialysate reservoir; a pump arranged to drive dialysate along the flow path; a sensing system arranged in the housing configured to sense one or more characteristics of the dialysate to produce an output; and a processor configured to observe said output to determine whether to switch on or off the pump such as based on pressure of the dialysate is above a predetermined value or a flow rate of the dialysate determined by the sensing system is zero. In a preferred embodiment, the one or more characteristics comprise at least one of turbidity, pressure and colour of the dialysate and the sensing system includes an optical sensor, a turbidity sensor and a pressure sensor.

### TECHNICAL FIELD

The present invention relates to a system for a dialysis treatment and, more particularly, but not exclusively, to a system for automatically controlling a peritoneal dialysis treatment.

### BACKGROUND

Renal diseases can be treated by kidney transplantation, haemodialysis, or peritoneal dialysis. Renal transplantation is not always possible for several reasons including availability of a matched transplant. Haemodialysis is an artificial maintenance therapy for end-stage renal disease. In haemodialysis, the blood is drawn from an artery passed through an artificial filter where mass transfer takes place with a counter-current dialysate flow, and pumped back to the venous system. Haemodialysis, though highly efficient is associated with numerous side effects including disequilibrium syndrome. It also has disadvantages including problems with availability of the filter, being stuck to the machine several hours with subsequent waste of time and money that lead to low quality of life.

Peritoneal dialysis is a lifesaving procedure for removing waste and excess water from the blood. It is used primarily to sustain the health of patients who have experienced renal failure. Peritoneal dialysis removes waste and excess water from the blood inside the body using the peritoneum as a natural semipermeable membrane. Waste and excess water move from the blood, across the peritoneal membrane, and into a special dialysis solution, called dialysate, in the abdominal cavity.

Peritoneal dialysis can be done using continuous ambulatory peritoneal dialysis. In particular, the dialysate is injected into the peritoneal cavity. After a long time, the retained fluid, which is now rich in waste metabolites, is drained. Injection and drainage of the liquid is a largely manual process, requiring the user to activate and deactivate a pump. The procedure is roughly out-of-control and the results are difficult to monitor. In addition, to achieve peritoneal dialysis a specific solution is introduced through a tube in the lower abdomen and then removed. The presence of the tube presents a risk of peritonitis due to the potential introduction of bacteria into the abdomen. For example, if the tube is not empty and clean, dialysate flowing through the tube will be mixed with the impurities in the tube. This can reduce the survival rate of patients.

It is generally desirable to overcome or ameliorate one or more of the above described difficulties, or to at least provide a suitable alternative.

### SUMMARY OF THE INVENTION

Presently disclosed is a system for dialysis, comprising:
a housing comprising a flow path along which dialysate passes between a subject and a dialysate reservoir;
a pump arranged to drive dialysate along the flow path;
a sensing system comprising an emitter arranged in the housing; and a detector arranged in the housing, wherein the emitter emits a signal towards the flow path and the detector detects the signal as reflected from the flow path.

The emitted and detector may for a single sensing unit of the sensing system. The emitter and detector may be disposed on opposite sides of the flow path.

The sensing system may be configured to sense one or more characteristics of the dialysate to produce an output, the system may then further comprise a processor configured to observe said output to determine whether to switch on or off the pump.

The one or more characteristics may comprise at least one of turbidity, pressure and colour of the dialysate.

The flow path may comprise a dialysate outflow, from the subject to the reservoir. The flow path may also comprise a dialysate inflow, from the reservoir to the subject.

The output produced by the sensing system may correspond to the signal emitted by the emitter as received at the detector.

The processor may be configured to determine, from the output, a density of the dialysate. The output may comprise two temporally spaced signals, the processor being configured to determine a change in density between the two temporally spaced signals. The processor may be also configured to determine, based on a rate of operation of the pump, at least one of a flow rate and volume of dialysate passing between the subject and dialysate reservoir.

The system may further comprise a non-transitory memory for storing respective ones of the received output, the flow rate, volume and/or density of the dialysate as determined by the processor. Said system may further comprise a transmitter, the processor transmitting, via the transmitter, a message corresponding to the respective ones of the output received from the detector, the flow rate, volume and/or density of the dialysate.

In use, a tube may be retained in the flow path. The tube may comprise a permeable section. The permeable section may be at least one of a light permeable section and a sound permeable section. The flow path may comprise a groove for receiving the tube. The system may further comprise a cover that is movable relative to the pump between a release condition, in which the tube can be inserted into or removed from the flow path, and an in-register condition in which the tube is held in register with the pump to enable the pump to drive dialysate within the tube along the flow path. The system may further comprise a button for releasing the cover when the cover is in the in-register condition, to enable the cover to move to the release condition.

The system may further comprises flow rate adjustment buttons for adjusting a flow rate of dialysate along the flow path.

Also disclosed herein is a method of controlling a system for dialysis, comprising:
using a pump to drive dialysate along a flow path arranged in a housing, wherein the dialysate passes between a subject and a dialysate reservoir;
using a sensing system arranged in the housing to sense one or more characteristics of the dialysate to produce an output; and
using a processor to observe said output to determine whether to switch on or off the pump.

The method may comprise using the processor to switch off the pump if pressure of the dialysate determined by the sensing system is above a predetermined value—it will be appreciated that, in terms of scope, being above a first threshold is the same as reaching (and potentially exceeding) a second threshold that is incrementally higher than the first threshold. Therefore, the term ‘above’ is intended to capture the first threshold and the second threshold which is a proxy for the first threshold. The method may also comprise using the processor to switch off the pump if a flow rate of the dialysate determined by the sensing system is zero.

### BRIEF DESCRIPTION OF THE DRAWINGS

Some embodiments will now be described, byway of non-limiting example only, with reference to the accompanying drawings in which:

FIG. 1 is a schematic illustration of a system or device for dialysis;

FIG. 2A is a diagram of draining out dialysate and FIG. 2B is a diagram of filling in dialysate;

FIG. 3 is a schematic illustration of a dialysis device comprising an emitter and a detector;

FIG. 4 is a schematic illustration of a dialysis device comprising a tube;

FIG. 5A is a schematic illustration of a dialysis device comprising a cover that is movable between a release condition, FIG. 5B is the device in an in-register;

FIG. 6 is a schematic illustration of a dialysis device comprising hooks for fixing the cover in said in-register condition;

FIG. 7 is a flow diagram showing a method used for controlling a device for dialysis.

FIG. 8 is a diagram showing a control logic;

FIG. 9 shows a process flow in a typical operation of a system for dialysis;

FIG. 10 is a flow diagram showing a method used for installing a device for dialysis;

FIG. 11 is a flow diagram showing a method used for installing a draining tubing;

FIG. 12 is a flow diagram showing a draining phase;

FIG. 13 is a flow diagram showing a method used for installing a flowing tubing;

FIG. 14 is a flow diagram showing an injection phase;

FIG. 15 is an overview of the treatment process; and

FIG. 16 is a more detailed process flow according with the treatment process of FIG. 15.

### DETAILED DESCRIPTION

Dialysis systems disclosed herein facilitate controlled peritoneal dialysis. The system monitors one or more parameters of dialysate. Those parameters can be used to determine the state of the dialysis process—e.g. whether there is an obstruction preventing adequate dialysate flow—and/or to determine whether the dialysis is being effective—e.g. whether the turbidity of the dialysate during the dialysis process indicates a health issue and so forth.

Embodiments of the dialysis system also enable the dialysis process to be stopped in the event that the parameter or parameters being monitored indicate an unsafe condition—e.g. a blockage in the tubing carrying the dialysate to the patient or from the patient. Further embodiments also control resumption of dialysis in the event that the unsafe condition resolves—e.g. line pressure increases/decreases to an acceptable level (e.g. within thresholds).

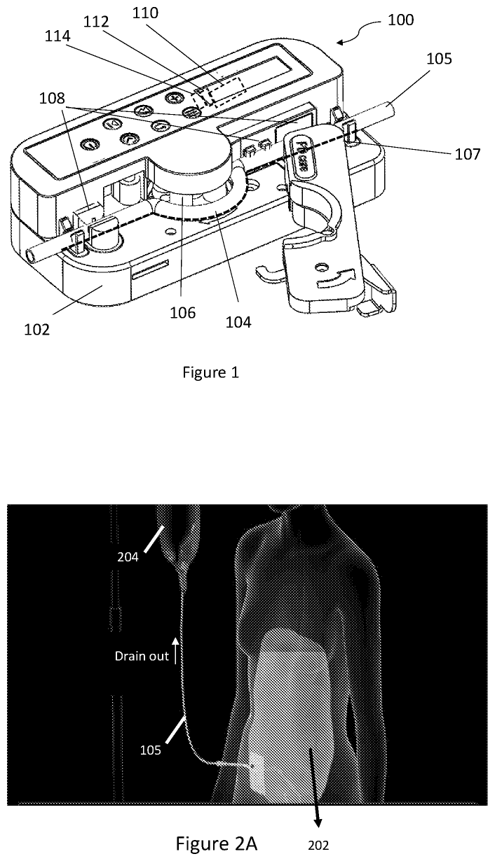

A device 100 for dialysis is described with reference to FIG. 1. The system will generally be a single device though may, if analysis of dialysate parameters is performed remotely, comprise a system of devices. For illustration purposes only, the system will hereafter be referred to as a device.

The device 100 broadly comprises a housing 102, pump 106, and sensing system 108. The housing 102 comprises a flow path 104 along which dialysate passes between a subject and a dialysate reservoir. When in use, the housing 102 encloses or captures a tube 105 positioned in the flow path 104. The tube 105 carries dialysate and the housing 102 ensures the dialysate flows through the device 100 along a predefined route—the flow path 104.

At discrete locations, or along its entire length, the flow path 104 maintains close cooperation with the tube 105, when in use—e.g. the flow path 104 has an internal surface the shape and size of which closely conform to an outer surface of the tube 105. To that end, the flow path 104 may comprise a groove for receiving the tube 402. The flow path 104 thus ensures the tube 105 is held in a predetermined position relative to the sensor(s) of the sensing system 108. This ensures accuracy, repeatability and robustness of measurements taken by the sensing system 108.

The pump 106 is arranged to drive dialysate along the flow path 104. The pump 106 may be a peristaltic pump, or in some embodiments may be a diaphragm pump or screw pump. The pump either drives dialysate into the patient (i.e. subject) or from the patient, depending on the flow direction in the tube 105 relative to the patient. In some embodiments, the pump is controlled by a processor housed within the housing.

The pump 106 may be bidirectional. This enables the tube 105 to be positioned in the flow path 104 and the pump 106 then activated in the desired direction of flow. This avoids the potential for a patient to connect the tube in the wrong direction, and thus pump towards a dialysate reservoir when attempting to deliver dialysate to the patient, or towards the patient when attempting to draw used dialysate from the patient. This also means the device 100 can be maintained in a common orientation relative to the patient, during both inflow and outflow of dialysate to/from the patient, by simply reversing the pumping direction.

The pump 106 may also, or alternatively, be a variable speed pump. This can enable greater pressure to be applied when attempting to clear blockages, or slower pumping to be used when nearing the end of the dialysis inflow or outflow operation. The speed of the pump, or the pressure applied thereby, may therefore vary along during the draining and/or filling cycle, such as varying according to a function or in increments depending on how complete the draining or filling cycle is. For example, during draining, the pump may switch on at a pressure of 1.1 atm, slow down if a pressure of 1.05 atm is detected and stop if a pressure of 1.01 atm is detected. Conversely, the pump may switch on during the filling phase at a pressure of 1.01 atm, slow down if a pressure of 1.05 atm is detected and stop if a pressure of 1.1 atm is detected.

The device 100 also includes a temperature control (i.e. heating) system or element 107—it is presently disposed along the flow path, beneath tube 105 and is thus shown in broken lines. The temperature control system 107 is arranged to warm dialysate during the filling phase. It may warm the dialysate on the fly or at the start of dialysis. This can increase patient comfort since the dialysate can enter the peritoneal cavity at approximately the temperature of the peritoneal cavity.

The sensing system 108 is arranged in the housing and is configured to sense one or more characteristics of the dialysate to produce an output. To this end, it comprises an emitter and detector from which the output is derived in a manner evident to the skilled person in view of present teachings.

The processor 110 (shown in broken lines since it is within the housing) is configured to observe the output of the sensing system 108. Observation comprises analysis of the output of the sensing system 108 for determining parameters of dialysate. This observation can then be used to determine whether to switch on or off the pump 106. In general, the system 100 will only be able to control switching of the pump to the OFF condition—i.e. no longer pumping dialysate. However, the system 100 may also switch the pump 106 back ON—e.g. if there was a blockage in view of which the pump 106 was switched OFF, but the sensing system 108 detects clearance of the blockage and thus switches the pump 106 back ON. Embodiments of the system 100 can therefore reduce the severe health risks associated with dialysis and issues that arise during dialysis.

The one or more characteristics measured by the sensing system 108 can include turbidity, pressure and colour of the dialysate. To that end, the sensing system 108 comprises at least one of a turbidity sensor, a pressure sensor and a colour sensor—these can be formed by the same, or respective pairs of, emitter and detector. The colour sensor and turbidity sensor are used to identify whether the dialysate is flowing through the flow path 104 (i.e. within tube 105), and to capture the transparency/clarity and/or colour of the dialysate. The pressure sensor is used for safety checks by detecting abnormal pressure of the flow path 104. Abnormal pressure can result, for example, from blockages.

As shown in FIG. 2A, the tube 105 can be used for dialysate outflow. Thus, the flow path 104 in such cases comprises a dialysate outflow (where the tube 105 enters the device 100), from the subject 202 to the dialysate reservoir 204. Similarly, the tube 105, and thus the flow path 104, can comprise a dialysate inflow (where the tube 105 enters the device 100), from the dialysate reservoir 204 to the subject 202, as illustrated in FIG. 2B.

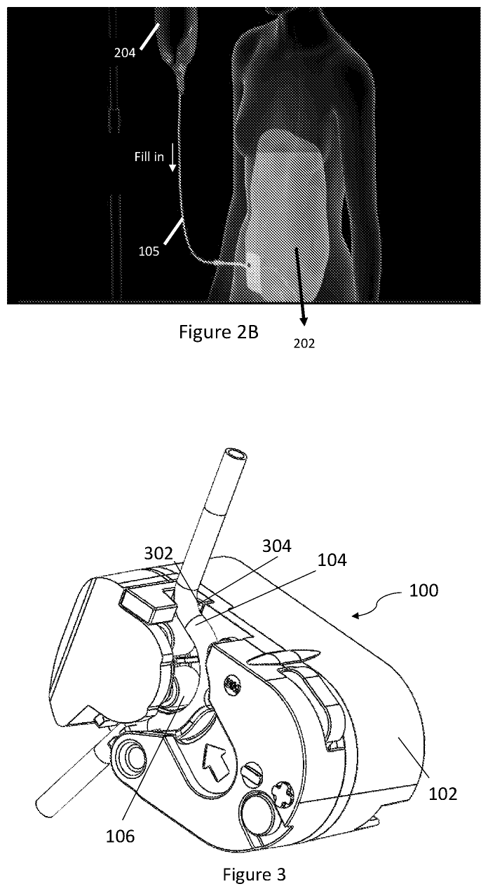

With reference to FIG. 3, the sensing system 108 comprises an emitter 302 and a detector 304, wherein the emitter 302 emits a signal into flow path 104 and light reflects back to the detector 304. The emitter 302 and detector 304 form a single device that emits light and detects reflected light—to that end, the light passes sufficiently into (and thus at least partially across) the flow path to enable the reflected light to bear the properties sought to be sensed, or is reflected by a mirror or reflector disposed opposite the emitter/detector relative to the flow path. The emitter 302 and detector 304 form components in a single sensing unit in the present embodiment. However, they may be separated in other embodiments. For example, the emitter 302 and detector 304 may be disposed on opposite sides of the flow path 104.

The output produced by the sensing system 108 may be corresponding to the signal emitted by the emitter 302 as received at the detector 304. For example, there the detector 304 detects light emitted from the emitter 302, that has passed through the dialysate, the sensing system 108 may detect turbidity or impurities in the dialysate. The degradation of dialysate during flow from the patient can indicate that dialysis is being performed effectively.

In one example, the emitter 302 is a light source and the detector 304 is a photo-detector, together forming an optical sensor or optical colour sensor. In another example, the emitter 302 and the detector 304 together form an ultrasound system. The optical sensor or ultrasound system can be used to detect the colour, transparency or the viscosity of dialysate. Detecting colour can help identify injury or illness—e.g. blood in the dialysate. Transparency can reflect the presence or otherwise of impurities. Transparency and viscosity can also reflect the density change of dialysate. The emitter 302 and the detector 304 may also together form the turbidity sensor. Through the turbidity sensor, it is easy to quantify the change of dialysate (e.g. change in clarity or transparency) and achieve detection of peritonitis at the early stage instead of using bare eyes.

The processor can thus be used to determine changes in density, turbidity, colour and other parameters of the dialysate. For example, the output of the sensing system 108 may comprise two temporally spaced signals, the processor 110 being configured to determine a change in density or other parameter(s) between the two temporally spaced signals.

The processor may be also configured to determine, based on a rate of operation of the pump 106, at least one of a flow rate and volume of dialysate passing between the subject 202 and the dialysate reservoir 204. Given the fixed tubing size and material, the flow rate of dialysate can be obtained if the rotation rate of motor is known. For continuous flow, the volume of dialysate can be calculated by multiplying the flow flux to the cross section surface area once the dialysate is starting to flow through the sensing system 108.

The device 100 further includes a non-transitory memory (reference 112 in FIG. 1) for storing respective ones of the received output, the flow rate, volume and/or density of the dialysate as determined by the processor. The device 100 is configured to measure and record the volume, density and flow rate of dialysate through edge computing, although other methods may be used as appropriate. The volume, density and flow rate of dialysate can be stored in the non-transitory memory. Through edge computing, the device 100 is able to compute all data and store the data on the memory card directly.

The device 100 further includes a transmitter (reference 114 in FIG. 1), the processor transmitting, via the transmitter, a message corresponding to the output received from the detector 304—i.e. the flow rate, volume and/or density of the dialysate. The patient can choose to transmit the data to a corresponding data recording App or upload to a cloud. Doctors and care givers can track the status of the patients using the App or cloud. The non-transitory memory also allows patients to easily carry the data around.

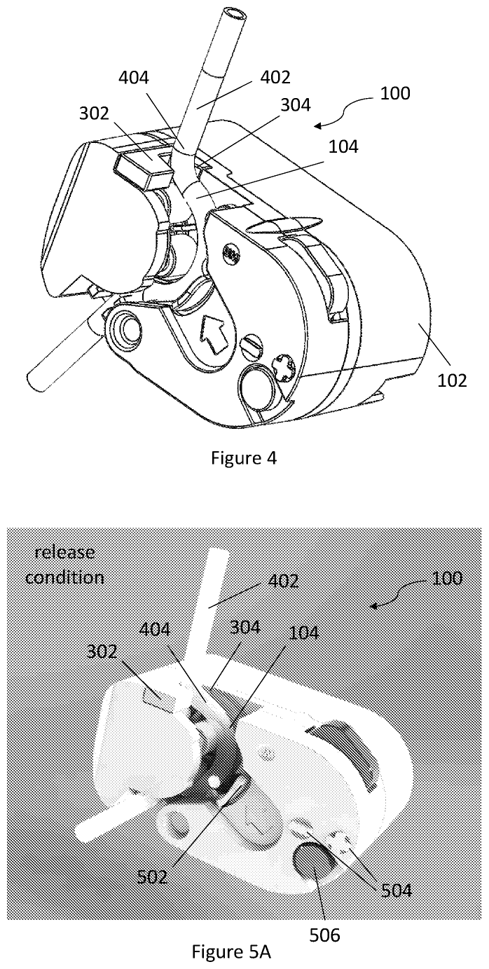

As shown in FIG. 4, while in use, a tube 402 of the dialysate reservoir is retained in the flow path 104. The tube 402 comprises a permeable section 404—presently light and sound permeable, although other embodiments may only be readily light or sound permeable, or may have other permeability. The permeable section 404 comprises the entire length of the tube 402. However, it will be appreciated that only a portion of the tube that is placed between the emitter 302 and detector 304 needs to be permeable.

The device 100, particularly the housing 102, includes a cover 502 that is movable relative to the pump between a release condition as shown in FIG. 5A and an in-register condition as shown in FIG. 5B. In the release condition, the tube 402 can be inserted into or removed from the flow path 104. In the in-register condition the tube 402 is held in register with the pump to enable the pump to drive dialysate within the tube 402 along the flow path 104.

The device 100 may further comprise a button 506 for releasing the cover when the cover is in the in-register condition, to enable the cover 502 to move to the release condition. The device 100 may also comprise flow rate adjustment buttons 504 for adjusting a flow rate of dialysate along the flow path, but adjusting the speed of the pump. It will be appreciated that the range of the speed rate of the dialysate may change due to different device placement conditions.

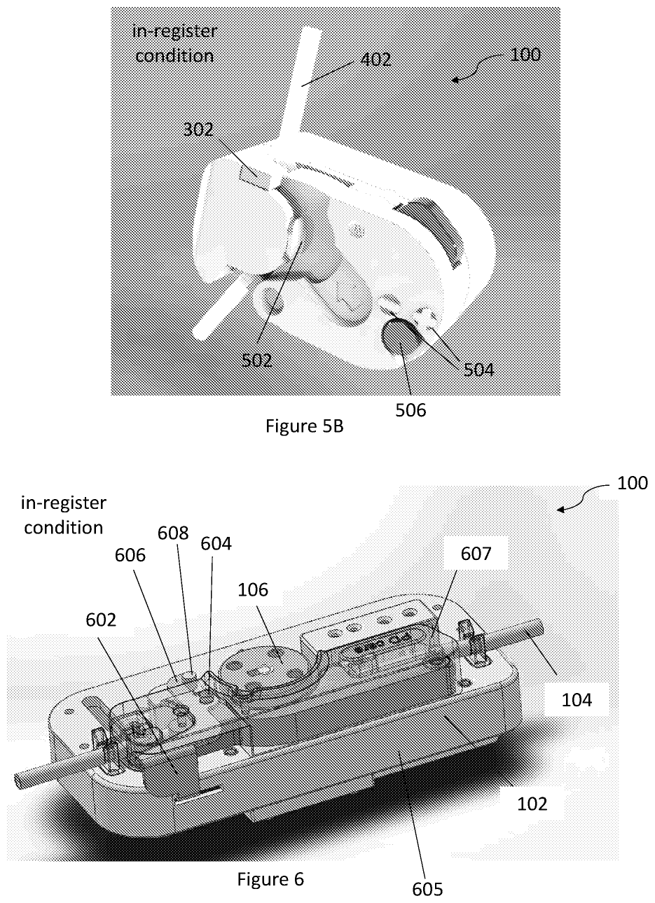

In the embodiment shown in FIG. 6, the button is replaced with a lever 602. The lever 602 is mounted to one part 605 of the housing 102. It is mounted to, and pivots around, pivot 604. pivoting around pivot 604 brings hook 606 around protrusion 608 provided on a second part 607 of the housing 102. This ensures the two parts 605, 607 of the housing 102 are in a predetermined relative position when the device is in the in-register condition. In addition, by having a flow path that closely conforms to the dialysate tube, the housing cannot close unless the tube is properly located in the flow path. When moving to the release condition, the lever 602 is simply rotated in the opposite direction to release protrusion 608.

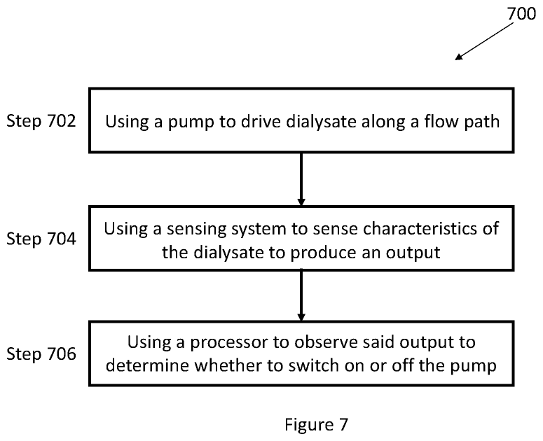

An example method 700 of controlling a device for dialysis is described with reference to FIG. 7. The method 600 broadly comprises:
Step 702: using a pump to drive dialysate along a flow path arranged in a housing, wherein the dialysate passes between a subject and a dialysate reservoir;
Step 704: using a sensing system arranged in the housing to sense one or more characteristics of the dialysate to produce an output; and
Step 706: using a processor to observe said output to determine whether to switch off the pump (and in some cases whether to switch the pump on).

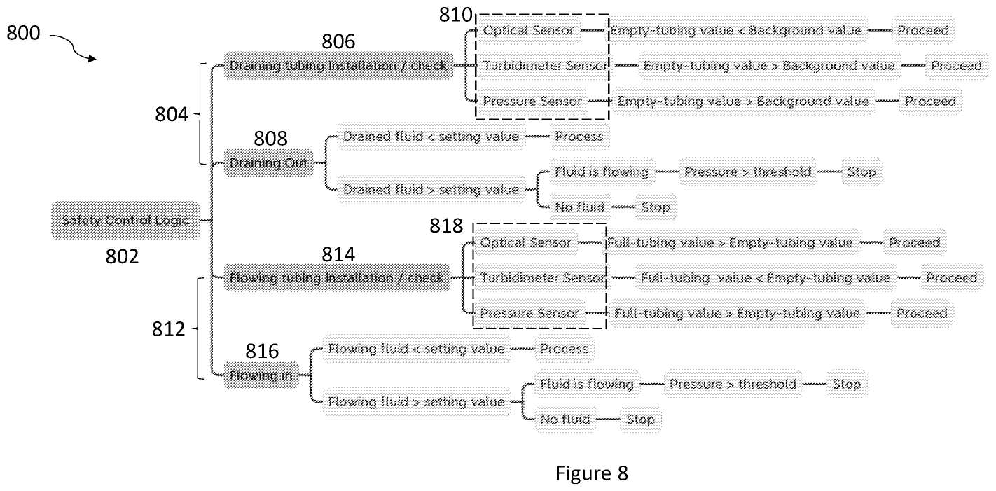

In the performance of Step 704 and Step 706, the system 100 employs control logic. An example of a control logic flow 800 is illustrated in FIG. 8. After activation of the pump at Step 702, to drive dialysate along the flow path, safety control logic check 802 is recursively performed. The check 802 can be performed during various processes and for various components of the system 100. In the illustrated example, during a draining operation 804 checks are performed by both analysing the dialysate via draining tube checks 806, and drainage process completion checks 808. The draining tube checks 806 involve using sensors of the sensing system—optical sensor, turbidimeter sensor and pressure sensor—810 to sense parameters of the dialysate.

The optical sensor checks properties of light passing through the draining tube. If the light passing through the draining tube has the same value (e.g. intensity or colour) as a background value, this indicates that the background is visible to the optical sensor and thus there is insufficient dialysate in the tube—e.g. blockage or completion of drainage. The system may then stop. Alternatively, if the value detected by the optical sensor is a value different from the background value (e.g. lower), this can indicate that there is dialysate in the tube and that draining should continue.

The turbidimeter sensor checks the turbidity of the contents of the tube. If the turbidity is greater than the background turbidity, this indicates that there is dialysate draining through the tube and that draining should thus continue. If the turbidity is equal to or lower than the background turbidity, this indicates that the process is complete or that there is a blockage. In either case, draining should cease.

The pressure sensor checks pressure in the tube, e.g. by detecting pump power required to drive fluid through the tube. If the pressure is greater than a background value then drainage is incomplete and pumping should continue. If the pressure is less than or equal to the background value, this indicates that there is a blockage or that pumping should otherwise cease since the draining operation is completed.

Further checks for drainage completion are based on volume per check 808. For check 808, the volume of fluid flowing through the tube is calculated. In the event that the drained volume is less than a threshold or predetermined set value, the drainage operation is not yet complete and should continue. If the drained volume is equal to or greater than the threshold or set value, the drainage operation should stop whether or not fluid is flowing through the tube. Drainage is complete.

Similarly, during a filling operation 812 checks are performed by both analysing the dialysate via filling tube checks 814, and filling process completion checks 816. The draining tube checks 814 involve using sensors of the sensing system—optical sensor, turbidimeter sensor and pressure sensor—818 to sense parameters of the dialysate.

The optical sensor checks properties of light passing through the filling tube, which may be the same tube as the draining tube. If the light passing through the filling tube has the same value (e.g. intensity or colour) as an empty tube value, this indicates that the tube is empty and filling should cease—e.g the dialysate reservoir has been depleted or emptied. The system may then stop. Alternatively, if the value detected by the optical sensor is a value different from the empty tube value (e.g. higher), this can indicate that there is dialysate in the tube and that filling should continue.

The turbidimeter sensor checks the turbidity of the contents of the tube. If the turbidity is less than the empty tube turbidity, this indicates that there is dialysate filling through the tube and that filling should thus continue. If the turbidity is equal to or greater than the empty tube turbidity, this indicates that the process is complete or that there is a blockage. In either case, filling should cease.

The pressure sensor checks pressure in the tube, e.g. by detecting pump power required to drive fluid through the tube. If the pressure is greater than an intra-abdominal pressure (or empty tube pressure) value then filling is incomplete and pumping should continue—assuming the height difference between the belly and the tubing is small, the pressure detected in the tubing can be used to indicate or infer the intra-abdominal pressure. If the pressure is less than or equal to the empty tube value, this indicates that there is a blockage or that pumping should otherwise cease since the filling operation is completed.

Further checks for filling completion are based on volume per check 816. For check 816, the volume of fluid flowing through the tube is calculated. In the event that the filled volume (i.e. the volume delivered to the subject) is less than a threshold or predetermined set value, the filling operation is not yet complete and should continue. If the filling volume is equal to or greater than the threshold or set value, the filling operation should stop whether or not fluid is flowing through the tube. Filling is complete.

Additional processes can be performed before filling or drainage. For example, before filling the subject 202 with dialysate from the dialysate reservoir 204, the processor will determine whether the tube 402 is empty or not. Similarly, before filling the dialysis reservoir 204 with the used dialysate from the subject 202, the processor will determine whether the tube 402 is empty or not. The pump 106 will not be switched on if the tube 402 is not empty. This is to keep the dialysate from mixing with the impurities in the tube. In one example, the sensing system 108 may comprise at least one of an optical sensor and a turbidimeter sensor, and can differentiate the transparency and the viscosity of liquid density, based on an empty tube or background transparency, which can reflect whether the tube 402 is empty or not. In another example, the sensing system 108 can also sense the pressure of the flow path to produce an output, and the processor can observe said output to determine whether the tube 402 is empty or not.

Thereafter, as mentioned above, when the subject 202 or dialysis reservoir 204 is being filled with the dialysis, the processor may determine to switch off the pump 106 if the pressure of the dialysate determined by the sensing system is above a predetermined value. The sensing system 108 can be used for safety checks by detecting abnormal pressure of the flow path.

With further reference to FIG. 7, the method 700 may also comprise using the processor to switch off the pump 106 if a flow rate of the dialysate determined by the sensing system 108 is zero. That is to say, the processor will be switched off if said dialysate is not flowing. Given the fixed tubing size and material, the flow rate of the dialysate can be obtained by determining the rotation rate of the pump 106. In some embodiments, the processor will determine whether all required dialysate has been filled into the subject 202 or dialysate reservoir 204 by calculating the volume of dialysate by multiplying the flow flux to the cross section surface area once the dialysate is starting to flow through the sensing system 108.

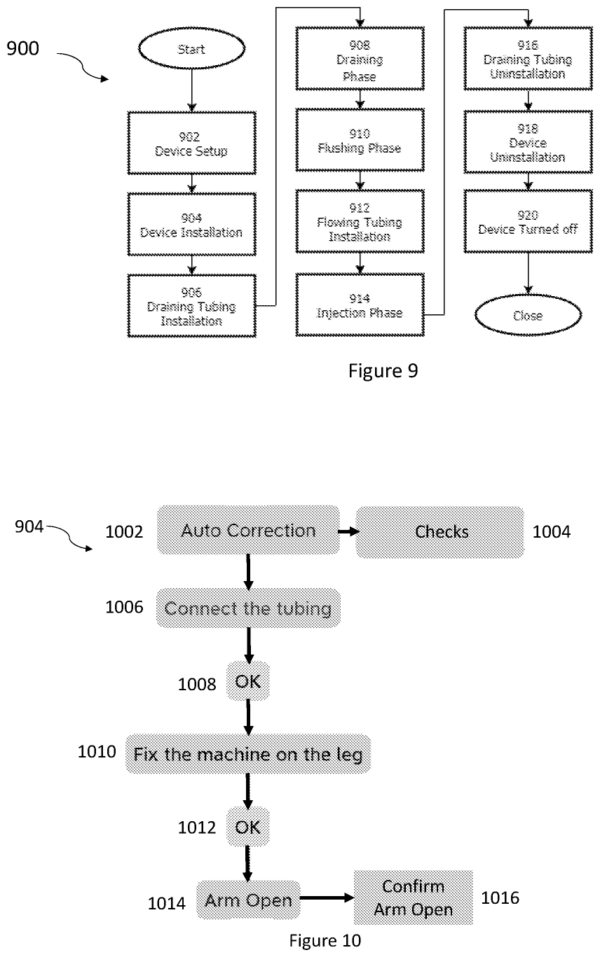

FIG. 9 shows a process flow 900 in a typical operation of the system 100. In general, the device is setup first (step 902), where a monitor or display on the system 100 may display the current state of the system 100 or components thereof (e.g. sensor readings). Then the device 100 is installed (step 904). The draining tubing is then installed (step 906) by inserting the tubing into the flow path of the device or system 100. The draining can then be commenced at step 908. Once the draining process is completed, the tubing is flushed (step 910). A flowing tube is then installed (step 912) in a similar manner to the drainage tube installed at step 906. The injection can then be commenced (step 914). After the injection process is completed, the tubing is removed from the housing (step 916), the machine or device 100 is removed from the league of the patient (step 918), the tube said is disassembled and the device is turned off.

At step 904, as illustrated in FIG. 10, auto correction (step 1002) may be performed first to research values back to 0. Step 904 also involve checks (step 1004) as discussed with reference to FIG. 7 and FIG. 8. The tubing is then connected (step 1006) by attaching the draining tubing to the transfer set in the patient's abdomen. While the system may automatically detect correct placement, a user may instead press a button (step 1008) to indicate that the tubing has been correctly placed and the cover has been closed. The device 100 is then fixed onto the leg of the subject (step 1010) which can again be automatically detected by the device 100 or can be confirmed by a button press (step 1012). The arm or cover of the device can then be open (step 1014) with the device 100 confirming (step 1016) that the arm is open and pumping should not be performed.

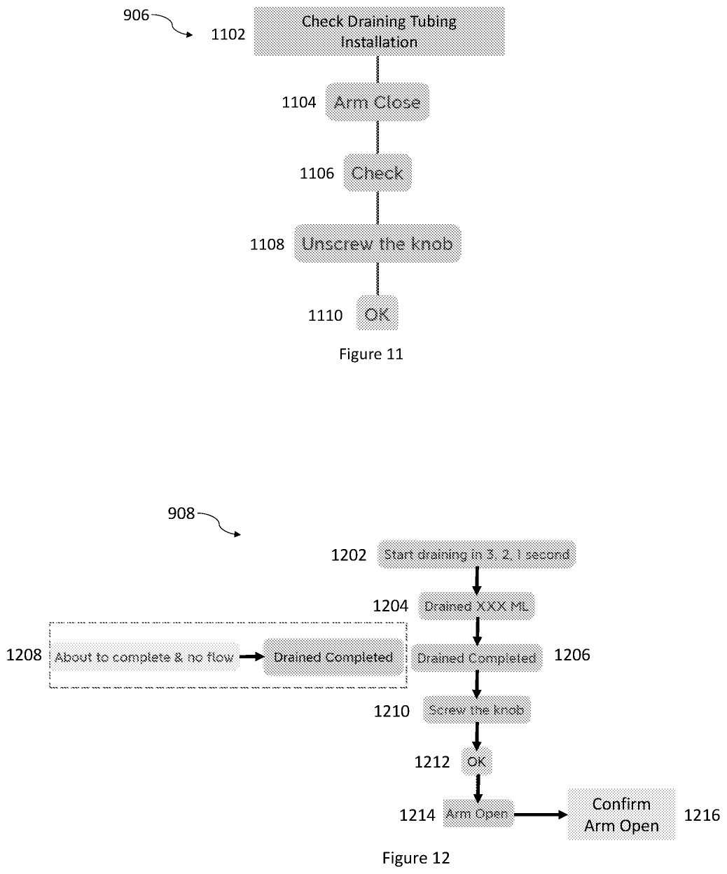

At step 906, as illustrated in FIG. 11, the draining tubing check is first initialised (step 1102) as discussed with reference to FIG. 8. The arm or cover is then closed (step 1104), the device then checks the status of the tubing, arm and/or any other components for which checks need to be conducted to ensure that draining can occur safely in a controlled manner (Step 1106). The device 100 automatically confirms that all is okay to proceed (e.g. that the tubing is been correctly installed) at step 1110, and then the draining process 908 will be stated.

At step 908, as shown in FIG. 12, the draining countdown can be commenced at step 1202. During draining process 908, measurements of the drain volume are calculated (step 1204) and a check is conducted to determine whether draining is complete (step 940). Once draining appears to be complete (step 942), e.g. no more dialysate is flowing through the tube, the system conducted check (step 1208) to determine whether draining should yet have been completed. The device 100 may automatically confirm completion, or the user may confirm completion (step 1212). The user then opens the arm or cover (step 1214), and the system monitors the release condition of the device 100 (step 1216).

At step 910, the tubing is then flushed. In some embodiments, the tubing may simply be discarded or the device 100 may be operable to flush the tubing prior to step 912—e.g. by connecting the tubing at one end to a cleaning fluid, water or saline solution and placing the other end at a drain.

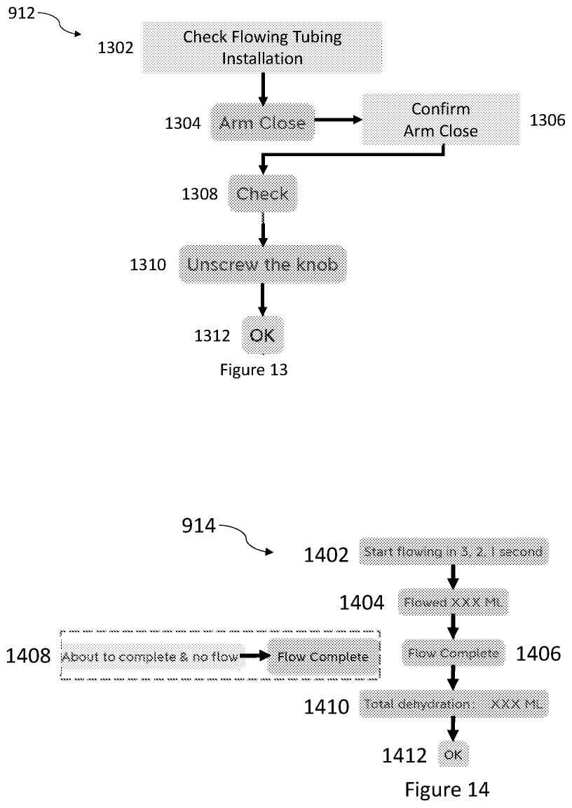

At step 912, as shown in FIG. 13, the user or device 100 then checks installation of the flowing tube (step 1302), the arm or cover is closed (step 960), the device 100 determines whether the arm has been properly closed (step 962), and the device 100 commences its checks of the flowing (filling) tube in line with processes (step 1308) described with reference to FIG. 7 and FIG. 8. Once the filling tube is in position, and the device has conducted its pre-filling operation checks, the user or the device confirms that filling can commence (step 1312).

At step 914, filling commences (step 1402) and the device 100 recursively detects the volume of fluid pumped into the subject (step 1404), using checks corresponding to FIG. 8. Once the device 100 detects completion (step 1406) via processes 1408 that correspond to those described with reference to FIG. 8—e.g. determining total dehydration by calculating the difference between fluid drained at step 914 versus injected or filled into the peritoneal cavity in the previous filling cycle, bearing in mind that incomplete drainage can indicate an accumulation of dialysate in the patient's body which can lead to edema—the filling process ends. The device 100 may automatically confirm completion, or the user may confirm completion (step 1412).

Measurements taken during draining and/or filling are saved (step 986), the arm or cover is then opened, the tubing is removed from the housing, be arm or cover is closed, the machine or device 100 is removed from the league of the patient, the tube said is disassembled and the device is turned off.

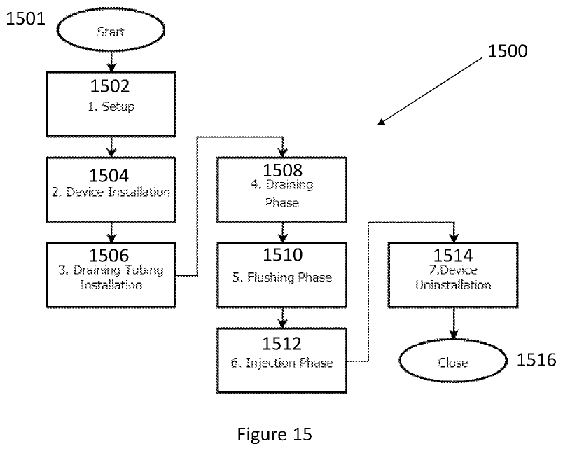

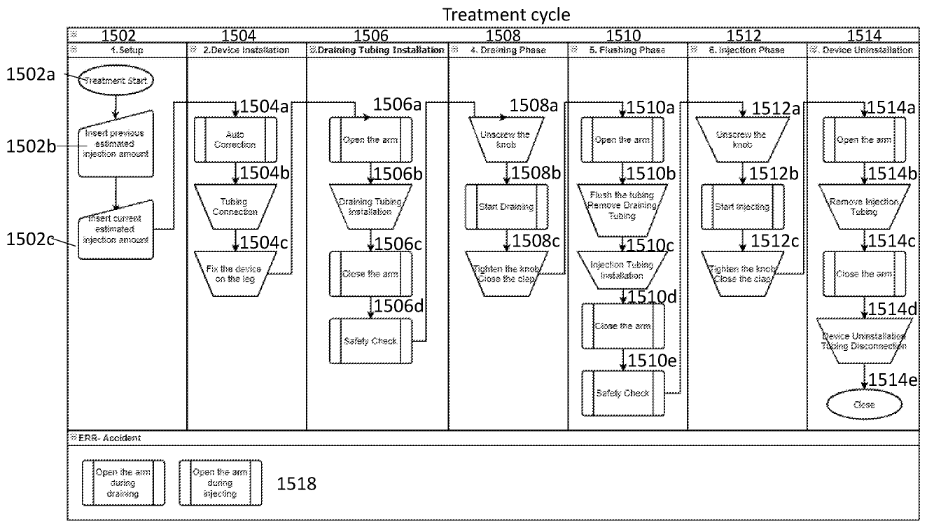

FIGS. 15 and 16 show a further process for operating a device such as device 100. The process 1500, as with all such processes, starts—Step 1501. The process 1500 then undergoes the following steps that are explained in greater detail in FIG. 16: set up Step 1502, device installation Step 1504, draining tubing installation Step 1506, draining phase Step 1508, flushing phase Step 1510, injection phase Step 1512, device uninstallation Step 1514 and a final closing or ending Step 1516.

With reference to FIG. 16: the set up Step 1502 comprises setting up the treatment on the device 100. This includes a treatment start input 1502a, that causes the device 100 to prompt the user for or to receive (e.g. from memory or a remote server) relevant inputs defining the treatment regime. These inputs comprise the acquisition or insertion by the user of a previous estimated injection amount (this may be alternatively stored in memory of the device 100) at 1502b, and the insertion of a current estimated injection amount or desired injection amount 1502c. The previous and current injection amounts may be extracted from memory or may be inputted using an interface involving, for example, the buttons mentioned above for other embodiments.

Device installation Step 1504 involves an auto-correction step 1504a. Auto-correction step 1504a performs a number of operations. Firstly, it may advise the user if the arm or housing has been properly closed. If not, it may prompt the user to properly close the housing. Once the device 100 has determined the housing is properly closed and secure, i.e. in the in-register condition without tubing in the flow path, one or more sensors may measure a respective background condition for use in comparative analyses at later steps in operation of the device 100. For example, the device may measure and store a background colour value, a background turbidity value, and a background pressure value.

To ensure the user properly connects all of the components of the system, device installation Step 1504 may also involve prompting the user to connect the tubing e.g. to a dialysate reservoir or to a drainage reservoir. Similarly, the device 100 may also prompt the user to attach or mount the device 100 to their body e.g. to the leg. Step 1504 then completes.

Draining tubing installation Step 1506 involves guiding the user in installing tubing in the device flow path 104. The device 100 may await opening, or may prompt the user to open, the arm of the housing 102 to gain access to the flow path—Step 1506a. After successful opening of the housing 102, the device 100 may then check whether draining tubing has been properly installed—Step 1506b—and prompt the user if the device 100 detects improper installation—e.g. clips or mounts in the flow path 104 failing to detect the presence of tubing, or sensors of the device 100 not detecting the presence of tubing such as by a slight alteration in the colour as sensed through a photodetector sensor. Upon detecting correct installation of the tubing in the flow path, the device may then prompt the user to close the housing, i.e. move it to the in-register condition—step 1506c.

At this point, the device 100 may undertake a safety check—Step 1506d. That safety check may involve polling the sensors to determine if they are operating properly and raising an error in the event that improper operation is detected. After running through the various sensor checks, the device 100 completes installation Step 1506.

After installation of the draining tubing at Step 1506, the device 100 can enter the draining phase Step 1508. This can involve the user unscrewing a knob to enable used dialysate to flow from the peritoneal cavity—Step 1508a. The device then enters a draining phase at Step 1508b, during which the device 100 continually monitors parameters/measurements such as whether the housing has moved from the in-register condition, whether the user's body is stable, if the pressure in the tubing 105 in the flow path 104 of the device 100 exceeds a first predetermined threshold that may be indicative of abnormal system operation, if the pressure is below a second predetermined threshold indicating that pumping speed should be controlled to control the pressure or the user should be prompted to check for air bubbles, if the pressure is below a third, low predetermined threshold indicating that there is a blockage in the tubing, and so on. For all errors (e.g. measurements indicating a pressure threshold has been exceeded, colour as measured by a photodetector use irregular or unexpected, the turbidity is too high and so on), or particular errors depending on the embodiment, the user may be prompted as to whether they wish to continue treatment or cease treatment.

Periodically, such as when the system has no detectable safety issues/errors, the drainage volume may be updated based on a flowmeter or other sensory output. The updated drainage volume may be displayed on the user interface so the user can monitor progress of the draining phase. This process will continue until draining completes, which may be automatically detected by the volume of used dialysate determined to have been drained from the peritoneal cavity. Upon completion of drainage the user is prompted to tighten the knob (e.g. on the transfer set connecting the device 100 to the peritoneal cavity) to prevent further fluid egress from the peritoneal cavity and confirm with the device 100 when this is been done.

Upon completion of the draining phase Step 1508, during which the peritoneal cavity is emptied of used dialysate, the system 100 enters the flushing phase at Step 1510. The flushing phase Step 1510 involves the steps of prompting the user to open the housing or arm (Step 1510a), close the housing or arm (Step 1510d) and perform safety checks (Step 1510e)—these steps are performed in the same manner as for draining tubing installation Steps 1506a, 1506c and 1506d. Between Steps 1510a and 1510d, the user is prompted to flush the draining tubing and remove it from the housing (Step 1510b) and install the injection tubing into the housing (Step 1510c). Step 1510c may be performed in the same manner as Step 1506b, including prompting the user in the event that the tubing is not properly installed in the flow path.

After flushing phase Step 1510, the device 100 enters the injection phase Step 1512. Injection phase Step 1512 involves Steps 1512a and 1512c for opening and closing the peritoneal cavity, that operate in the same manner as Steps 1508a and 1508c. Between Steps 1512a and 1512c, the device 100 commences injection at Step 1512b. During injection step 1512b the device continually monitors if the arm or housing moves away from the in-register condition, e.g. towards the open condition, if the user's body is stable, and if the amount of dialysate injected into the peritoneal cavity is at or has exceeded an estimated predetermined required amount of dialysate.

If the device moves away from the in-register condition or the user's body is unstable, an alarm may be raised and the user may be prompted to press a button to cancel the alarm or close the housing. If the amount of dialysate injected into the peritoneal cavity has reached the predetermined amount, the device 100 completes Step 1512. If the amount of dialysate injected into the peritoneal cavity has not yet reached the predetermined amount, the device 100 checks whether the flow path or tubing pressure is within predetermined, desirable limits. If the pressure is higher than a predetermined limit, the system may either cease injection or may prompt the user to confirm whether they wish to continue with injection. If the user elects to stop injection, the device 100 may continue to check the pressure in the tubing or flow path and periodically prompt the user to confirm they still wish for injection not to continue. If the user elects to continue injection, or the pressure has not exceeded the predetermined threshold, the system checks whether the pressure is above a second, low threshold. If the pressure in the flow path or tubing is smaller than that of a previous cycle—the device 100 cycling through the present Step 1512 so repeatedly perform all safety checks and update volume of dialysate extracted from or delivered to the peritoneal cavity—the system may, based on pressure readings, determine whether the housing has moved from the in-register condition or if an air bubble exists in the tube or flow path. If the event of detection of an error—i.e. the housing moving from the in-register condition or on detection of an air bubble—the device may either prompt the user to close the housing or check for air bubbles and/or terminate injection. In some cases, in the event that the pressure sensor measurement indicates the presence of an air bubble, the system may periodically check the pressure. If that periodic check indicates the issue has resolved, the device 100 may continue injection. If that periodic check indicates the issue has not resolved, the device 100 may raise an alert and either prompt the user to indicate whether they wish to cease or proceed with injection, or may automatically cease injection (e.g. shut off the pump).

During the injection Step 1512b, the device 100 may repeatedly cycle through the above process. For each cycle, the device may update the amount of injected dialysate as measured by the flowmeter or other sensor in the device 100.

On completion of the injection phase Step 1512, the device enters the device uninstallation phase Step 1514. As with Steps 1510a and 1510d, the device prompts the user to open the arm or housing at Step 1514a to enable the injection tubing to be removed from the flow path, and to close the housing at Step 1514c. Between Steps 1514a and 1514c, the device 100 may prompt the user to remove the injection tubing at Step 1514b. The device then prompts the user to disconnect the tubing from the dialysate reservoir or bag at Step 1514d and then ends the treatment cycle—Step 1514e.

For completeness, field 1518 in FIG. 16 shows a subset of the errors that can arise during the treatment cycle. The area shown are the arm or housing opening during draining or injection, again to the housing moving away from the in-register condition. Many other errors will arise based on sensor measurements as mentioned above.

It will be appreciated at various of the processes performed by the user may be automated by the device 100, such as checking back to the tubing is correctly installed in the housing. All such modifications are intended to fall within the present disclosure and will be evident to the skilled person in view of present teachings.

It will be appreciated that many further modifications and permutations of various aspects of the described embodiments are possible. Accordingly, the described aspects are intended to embrace all such alterations, modifications, and variations that fall within the spirit and scope of the appended claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavor to which this specification relates.

1. A system for dialysis, comprising:
a housing comprising a flow path along which dialysate passes between a subject and a dialysate reservoir;
a pump arranged to drive dialysate along the flow path;
a sensing system comprising an emitter arranged in the housing; and
a detector arranged in the housing,
wherein the emitter emits a signal towards the flow path and the detector detects the signal as reflected from the flow path.

2. The system of claim 1, wherein the sensing system is configured to sense one or more characteristics of the dialysate to produce an output, the system further comprising a processor configured to observe said output to determine whether to switch on or off the pump.

3. The system of claim 1, wherein the one or more characteristics comprise at least one of turbidity, pressure and colour of the dialysate.

4. The system of claim 1, wherein the flow path comprises a dialysate outflow, from the subject to the reservoir.

5. The system of claim 1, wherein the flow path comprises a dialysate inflow, from the reservoir to the subject.

6. The system of claim 2, wherein the output produced by the sensing system corresponds to the signal emitted by the emitter as received at the detector.

7. The system of claim 2, wherein the processor is configured to determine, from the output, a density of the dialysate.

8. The system of claim 7, wherein the output comprises two temporally spaced signals, the processor being configured to determine a change in density between the two temporally spaced signals.

9. The system of claim 1, wherein the processor is configured to determine, based on a rate of operation of the pump, at least one of a flow rate and volume of dialysate passing between the subject and dialysate reservoir.

10. The system of claim 2, further comprising a non-transitory memory for storing respective ones of the received output, the flow rate, volume and/or density of the dialysate as determined by the processor.

11. The system of claim 2, further comprising a transmitter, the processor transmitting, via the transmitter, a message corresponding to the respective ones of the output received from the detector, the flow rate, volume and/or density of the dialysate.

12. The system of claim 1, wherein, in use, a tube is retained in the flow path.

13. The system of claim 12, wherein the flow path comprises a groove for receiving the tube, the system further comprising a cover that is movable relative to the pump between a release condition, in which the tube can be inserted into or removed from the flow path, and an in-register condition in which the tube is held in register with the pump to enable the pump to drive dialysate within the tube along the flow path.

14. The system of claim 13, further comprising a lever for releasing the cover when the cover is in the in-register condition, to enable the cover to move to the release condition, and for locking the cover in place in the in-register condition.

15. The system of claim 1, further comprising flow rate adjustment buttons for adjusting a flow rate of dialysate along the flow path.

16. A method of controlling a system for dialysis, comprising:
using a pump to drive dialysate along a flow path arranged in a housing, wherein the dialysate passes between a subject and a dialysate reservoir;
using a sensing system arranged in the housing to sense one or more characteristics of the dialysate to produce an output; and
using a processor to observe said output to determine whether to switch on or off the pump.

17. The method of claim 16 comprises using the processor to switch off the pump if pressure of the dialysate determined by the sensing system is above a predetermined value.

18. The method of claim 16 comprises using the processor to switch off the pump if a flow rate of the dialysate determined by the sensing system is zero.
